(19) United States
(12) Patent Application Publication
(10) Pub. No.: US 2011/0166628 A1
(43) Pub. Date: Jul. 7, 2011

(54) SYSTEM, METHOD AND DEVICE FOR MEDICAL DEVICE DATA PROCESSING AND MANAGEMENT

(76) Inventor: Praduman D. JAIN, Fairfax, VA (US)

(21) Appl. No.: 12/652,368

(22) Filed: Jan. 5, 2010

(51) Int. Cl. A61N 1/08; G06F 15/16

(52) U.S. Cl. 607/60; 709/217

(57) ABSTRACT

A system, method, and computer readable medium for monitoring and controlling medical devices having a data processing server configured to receive from a user instructions relating to a medical device. The system also includes an application hosting device configured to receive the instructions from the data processing server. The application hosting device is further configured to modify the instructions for transmission to the medical device. The medical device is configured to receive the modified instructions and perform the modified instructions. Another aspect provides an application hosting device that captures medical device data and includes a processor and a connection unit configured to establish a link to exchange information. The application hosting device is configured to connect to a plurality of medical devices simultaneously. The plurality of medical devices comprise at least one master device and at least one slave device.

### FIELD

The present invention relates to a system, method, and device for medical device data processing and management.

### BACKGROUND

Medical devices have become more complex due to the advent of new technologies, such as improved computing power, improved network communications, and faster and more compact storage media. Medical devices are typically equipped with processors or microprocessors and storage media such that readings obtained from patients or other users may be stored in the medical devices and transmitted to another device or system. Many of the medical devices are worn on or in the body and do not have any direct connectivity for data gathering and analysis. Body area networking may be used to connect some of these medical devices to other devices, for example, computer devices. Additionally, medical devices may be part of networks that enable the exchange of information between various institutional information systems and/or other medical devices.

Medical device management (MDM) refers to tools intended to distribute applications, data and configuration settings to medical devices. The purpose of MDM is to optimize the functionality and security of medical device communication systems while minimizing cost and downtime.

### SUMMARY

As the functionalities of medical devices grow at an increasing rate, configuring and maintaining the services and features of the medical devices are becoming more complex and time-consuming. Configuring these devices may be difficult and confusing for typical users.

Additionally, massive amounts of data may be generated and transmitted to or from such medical devices. Thus, there is a need to be able to organize patient data and to be able to associate the data with certain patients and certain medical devices. Additionally, as the complexity of medical devices and the amount of data generated increases, it may be necessary for the doctors, clinicians, or health care providers to be able to remotely monitor and control medical devices and to receive and analyze information from the medical devices.

As such, there is a need, for example, for a medical device communications system that is able to efficiently process and transmit data and instructions to and from the medical devices.

An aspect provides a system for monitoring and controlling medical devices having a data processing server configured to receive from a user instructions relating to a medical device. The system also includes an application hosting device configured to receive the instructions from the data processing server, the application hosting device further configured to modify the instructions for transmission to the medical device. The medical device is configured to receive the modified instructions and perform the modified instructions.

Another aspect provides a system for delivering medical device data. The system has an application hosting device configured to receive data from a medical device using a first network. The application hosting device is further configured to process the data and send the data via a second network, wherein the first network is different from the second network.

Another aspect provides an application hosting device to capture medical device data. The application hosting device includes a processor and a connection unit configured to establish a link to exchange information. The application hosting device is configured to connect to a plurality of medical devices simultaneously, wherein the plurality of medical devices comprise at least one master device and at least one slave device.

Another aspect provides a method of monitoring and controlling a medical device. The method includes receiving, from a data processing server, instructions relating to a medical device and identifying the medical device associated with the instructions. The method further includes modifying the instructions for transmission to the medical device and transmitting the modified instructions to the medical device.

Another aspect provides a computer-readable storage medium having computer-executable code for execution by a processing system to control a medical device. The code is configured to identify the medical device associated with received instructions. The code is further configured to modify the instructions for transmission to the medical device and to transmit the modified instructions to the medical device.

These and other aspects of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood that the drawings are for the purpose of illustration and description only and are not a limitation of the invention. In addition, it should be appreciated that structural features shown or described in any one embodiment herein can be used in other embodiments as well. As used in the specification and in the claims, the singular form of “a”, “an”, and “the” include plural referents unless the context clearly dictates otherwise.

### BRIEF DESCRIPTION OF THE DRAWINGS

FIG. 1 is a block diagram of a medical device data capture and processing system 10 in accordance with an embodiment;

FIG. 2 is a block diagram of the modules of an application hosting device in accordance with an embodiment;

FIG. 3 is a block diagram illustrating the architecture of the application hosting device in accordance with an embodiment;

FIG. 4 is a block diagram illustrating the master and slave relationships of medical devices with the application hosting device;

FIG. 5 is a block diagram showing the application and web services in accordance with an embodiment;

FIG. 6 is an exemplary signaling diagram for a method of providing bi-directional communication in accordance with an embodiment;

FIG. 7 is a flowchart illustrating a method of capturing data from medical devices and packaging the data for transmission in accordance with an embodiment;

FIG. 8 is a flowchart illustrating a method for initializing and preparing connection for capturing data in accordance with an embodiment;

FIG. 9 is a flowchart illustrating a method for capturing data and processing the data in accordance with an embodiment;

FIG. 10 is a flowchart illustrating a method for parsing and validating data by the application hosting device in accordance with an embodiment;

FIG. 11 is a flowchart illustrating a method for enabling traceability of data in accordance with an embodiment;

FIG. 12 is a flowchart illustrating a method for processing packaged data by a data processing server;

FIG. 13 is a flowchart illustrating a method for submitting data to third party applications in accordance with an embodiment;

FIGS. 14A-14C are block diagrams showing various combinations of and connections for the medical device, application hosting device, and data processing server;

FIG. 15 is a signaling diagram showing communication between a medical device and an application hosting device; and

FIG. 16 is a flowchart illustrating a method for implementing a master and slave connection in accordance with an embodiment.

### DETAILED DESCRIPTION

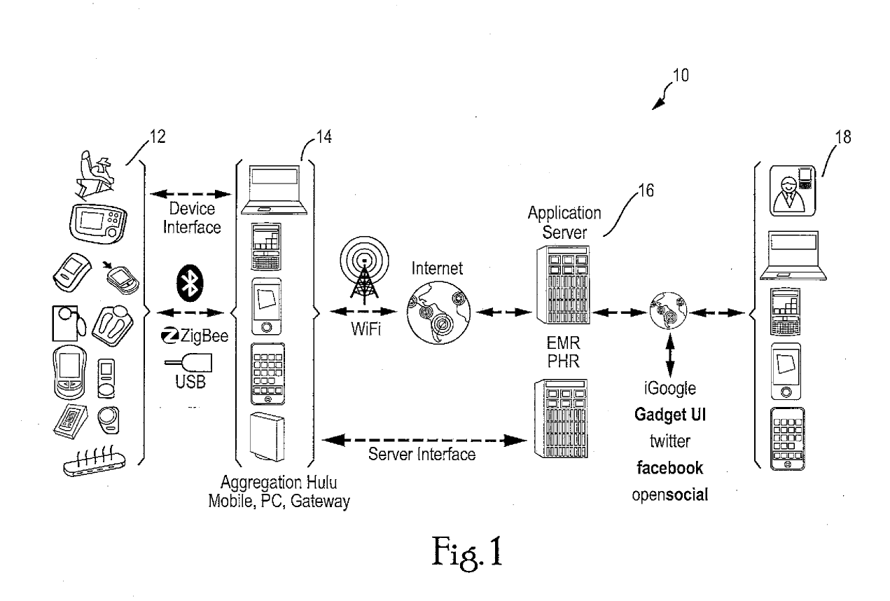

FIG. 1 shows a medical device data capture and processing system 10 in accordance with an embodiment of the invention. The processing system 10 includes at least one medical device 12. The medical device 12 may be, for example, a physiological monitor (e.g., blood pressure, EEG, ECG, heart rate, pulse oximeter, or other monitor), drug delivery device (e.g., a pill bottle or dispenser, IV, etc.), a therapy device (e.g., a treadmill, stationary bike, weight system, etc.), a pump, or any other device used in health care. As used herein, the terms “medical device” and “medical devices” encompass a medical device, a health or healthcare device, or any device that monitors a user or promotes the wellbeing of a user. The medical device 12 may be operatively connected to a user in various ways. For example, the medical device may be placed inside the body of the user (e.g., a pacemaker), on the body (e.g., a blood pressure gauge), and/or around the body (e.g., a weight scale). In this embodiment, the medical device 12 is in communication with an application hosting device 14. The application hosting device 14 may be a cellular telephone, a smart phone, a pager, a personal digital assistant (PDA), a portable computer, or any other electronic device capable of receiving information from the medical device 12. The application hosting device 14 may also include a user interface, such as one or a combination of a touch screen, screen, and/or a mouse pointer. The application hosting device 14 is not limited to the mobile or portable computing devices listed above, and may also encompass any computing device, for example, a personal computer, a desktop computer, or other computing device.

The processing system 10 includes a data processing server 16 configured to receive data from the medical device 12, either directly or indirectly via the application hosting device 14. The data from the medical device 12 may comprise measurement data (such as data relating to the measured physiological characteristic(s) of a user using the medical device 12), device status information, device identity information, time and date information, and/or other information relating to the status or condition of the medical device 12 and/or the user. Measurement data may include health data, vital signs, systolic pressure data, diastolic pressure data, pulse data, and/or other types of data relating to the user.

The various components of the data processing server 16 may operate on a software operating system such as Microsoft Windows, Linux, Sun Solaris, Apple Macintosh OS, and/or UNIX operating system. In an embodiment, the application hosting device 14 and/or the data processing server 16 may include a database. The database may be based on a relational database, such as a MySQL, Microsoft SQL Server, or Oracle database. It is also contemplated that the number of the components of the system 10 may vary. The components shown in FIG. 1 can be implemented with any combination of hardware or software, including software executed by multiple computer systems or servers.

It is also contemplated that the data processing server 16 may be associated with a web client through a device, such as a personal computer, or any other electronic device capable of receiving information from a medical device 12. Thus, some of the descriptions herein with respect to the application hosting device 14 may be applicable to the web client. The application hosting device 14 or the web client may be configured to submit requests/instructions and receive data to and from the data processing server 16 via a mobile phone network, the Internet, a mobile phone network employing the wireless application protocol (WAP), a local area network (LAN), a wide area network (WAN), a wireless local area network (WLAN, also known as WiFi network or IEEE 802.11x network), a telecommunications network (2G, 3G, 4G, GSM, CDMA, GPRS, EDGE, EVDO, HSPD), an 802.16 network (WiMAX), IEEE P1901 BPL (Broadband over Power Lines), a facsimile network, a satellite network, a RF network, or other communication means.

The medical device 12 and the application hosting device 14 may be in communication with one another via a protocol such as RF, Bluetooth, ZigBee (or other variation of the IEEE 802.15 protocol), Near Field Communication (NFC), IEEE 802.11 (or any variation thereof), IEEE 802.16 (WiMAX or any other variation), a cellular/wireless/cordless telecommunication protocol, a wireless data communication protocol such as Wireless USB, or any other communication protocol. In some embodiments, the medical device 12 and the application hosting device 14 may be in communication with one another via a wire or other physical link, for example, via . Ethernet or USB connection. In such an embodiment, the medical device 12 and the application hosting device 14 may have a wire/cabled local device interface that may include a jack, plug, socket, receptacle, or other interface. As mentioned above, in one embodiment, the medical device 12 and the application hosting device 14 may communicate with one another via Bluetooth. The medical device 12 and the application hosting device 14 may include Bluetooth interfaces that may be implemented using a combination of hardware, software, and/or firmware. In some embodiments, the Bluetooth interfaces may include an RF front end, a radio module, a Bluetooth transceiver, and/or an RF antenna. A wireless (RF) radio module may include a wireless receiver module integrated with a wireless transmitter module. An application hosting. device 12 may be equipped with a Bluetooth adapter and/or may be equipped with an Infrared Data Association (IrDA) adapter.

Communication between the server 16 and the application hosting device 14 or the web client may be made according to the HyperText Transfer Protocol (HTTP) and/or the Simple Object Access Protocol (SOAP). In some embodiments, the data may be sent in the Extensible Markup Language (XML) format through the HTTP protocol. Details of this process will be described later.

The application hosting device 14 and the medical device 12 may have a portable power supply, for example, a battery, or it may require connection to a power grid. The application hosting device 14 and the medical device 12 may include one or multiple processors and memory. In some embodiments, the processor may be a microprocessor, a controller, or a microcontroller. The processor may be implemented as a combination of computing devices, for example, a combination of digital signal processor and a microprocessor, or any other configuration. In one embodiment, the application hosting device 14 provides a graphical user interface. The user interface may include a display and an input device, which may be a touch screen, a mouse pointer, a keyboard, or other device. The input device may be integral with the display or may be separate and configured to interact with the display. The user interface enables a user, such as a patient, doctor, clinician, or other health care provider, or anyone using the application hosting device 14, to interact with the application hosting device 14 such that the user can, for example, input requests for information from or input instructions to be submitted to, the medical device 12.

In an embodiment, the application hosting device 14 and/or data processing server 16 may be connected (e.g., via the Internet or other communication network or mechanism) to one or more third party applications or systems 18. Such third party application or systems 18 will be described in further detail below.

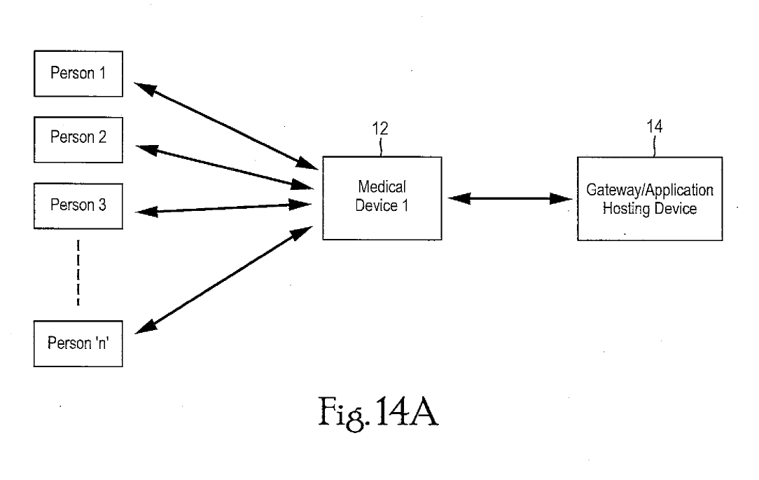

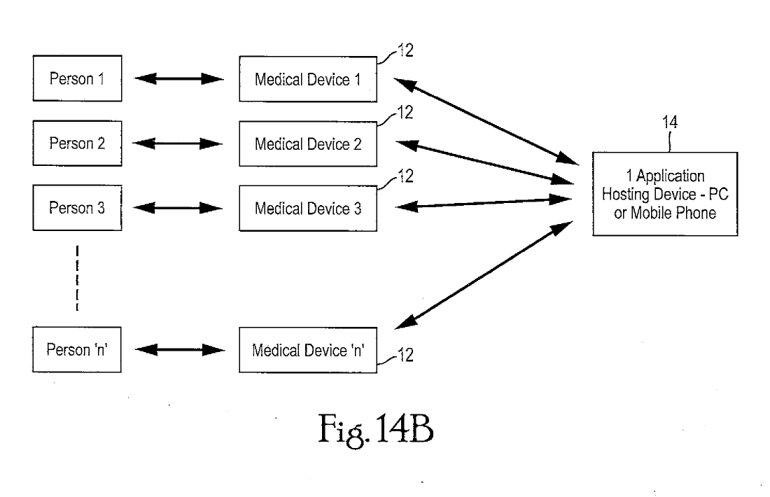

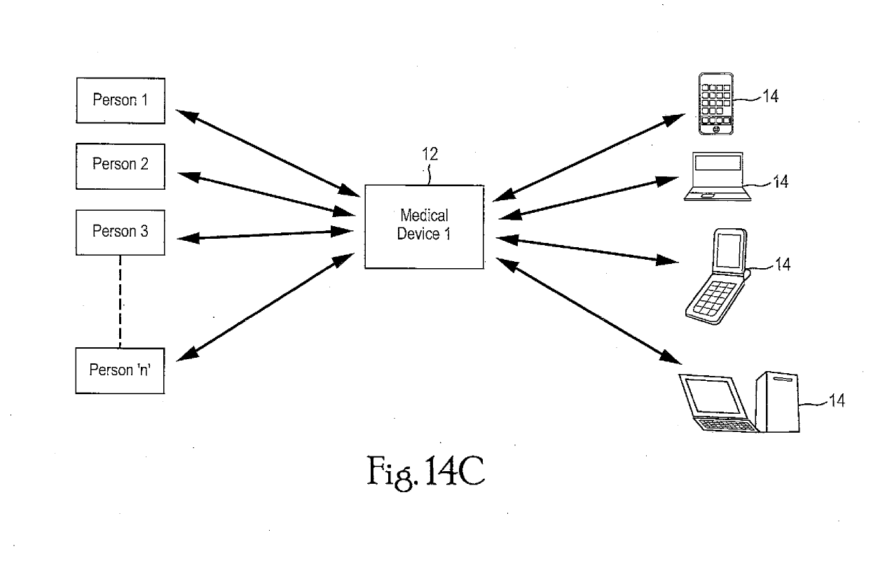

It is contemplated that in some embodiments, one medical device 12 may be associated with several users (see FIG. 14A). In addition, the one medical device may be associated with a plurality of application hosting devices 14 (see FIG. 14C). In some embodiments, a plurality of users may be associated with a plurality of medical devices 12 and a plurality of application hosting devices 14. For example, one user may be associated with one medical device 12 and the one medical device may be associated with one application hosting device 14. In another example, a plurality of medical devices 12 may be associated with a single application hosting device 14. In other embodiments, each user of a plurality of users may be associated with a medical device 12 and all of the medical devices 12 may be associated with a single application hosting device 14 (see FIG. 14B). Therefore, it is contemplated that the combination of and, relationship among the users, medical devices 12, and the computing devices 14 may vary.

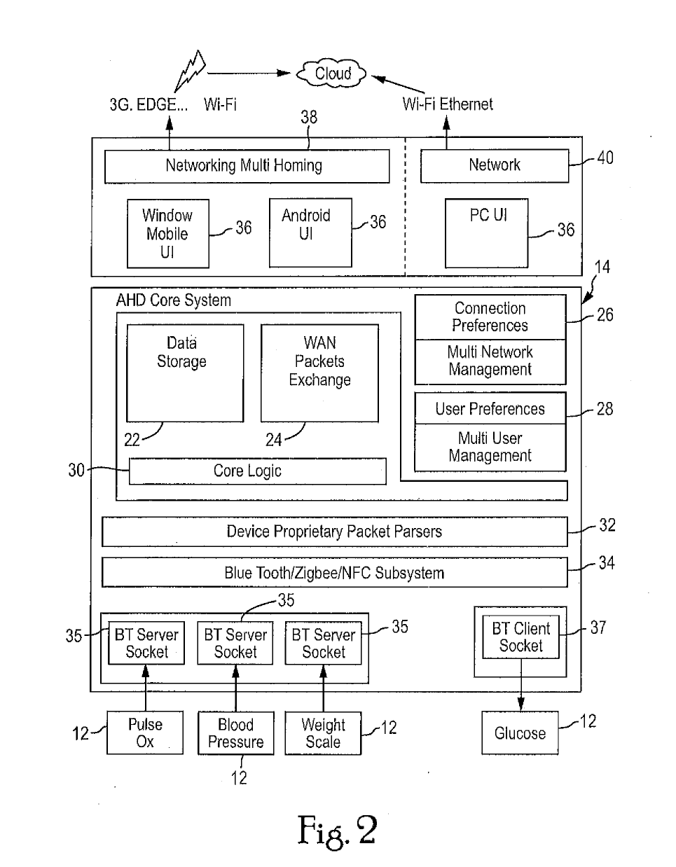

FIG. 2 shows an embodiment of the application hosting device (AHD) 14 that may be used with multiple users and/or multiple medical devices 12. In this embodiment, the application hosting device 14 includes a data storage module 22 configured to store data for retrieval. It may write/read the data using a variety of methods. The application hosting device 14 may also include a WAN packets exchange module 24 configured to process data packets that are exchanged through a WAN network. The core logic module 30 of the application hosting device 14 is configured to implement the logic for the operation of the application hosting device 14 within the system 10. As noted above, the application hosting device 14 may be used with multiple users and multiple networks. As such, the connection preferences module 26 and the user preferences module 28 may be configured to manage the various multiple networks and the multiple users, respectively. The settings for each user may be stored in the user, preferences storage module 28. The connection parameters may be stored in the connection preferences storage module 26. The device proprietary packet parsers module 32 is configured to parse data packets, such as the data packets received from the medical devices 12. The application hosting device 14 may also include a PAN subsystem module 34, which may be configured to support technologies such as Bluetooth, Zigbee, NFC, and/or others. In this embodiment, the application hosting device 14 uses Bluetooth to connect to master and slave medical devices 12. For example, the application hosting device 14 may open a server socket (e.g., a Bluetooth server socket 35) for a master medical device 12, which may be, for example, a pulse oximeter, a blood pressure monitor, or a weight scale. In addition or alternatively, the application hosting device 14 may open a client socket (e.g., a Bluetooth client socket 37) for a slave medical device 12, such as a glucose monitor. The PAN subsystem module 34 may store the pairing information for the application hosting device 14 and a medical device 12. The medical device 12 and the application hosting device 14 may need to be paired to communicate with each other. The pairing process may be triggered automatically the first time an application hosting device 14 receives a connection request from a medical device 12 it is not yet paired with, or vice versa. Once a pairing has been established, the information is stored in the application hosting device 14 and the medical device 12 so that they can then connect to each other without user intervention. When desired, the pairing relationship can later be removed by the user. In one embodiment, the pairing between a medical device 12 and the application hosting device 14 may be made via Secure Simple Pairing (SSP).

The application hosting device 14 may include a user interface 36 configured to enable a user to input information and for the application hosting device 14 to output information. The user interface 36 may be presented using any computing device (e.g., phone, personal digital assistant, PC, etc.), including any one or combination of a Microsoft Windows Mobile user interface, a Google Android user interface, iPhone user interface, Java user interface, Symbian user interface and/or a PC user interface, although other types of user interfaces are contemplated. The application hosting device 14 may also include a network multi homing module 38 configured to search for available networks by which it can communicate with the data processing server 16. For example, in one embodiment, the application hosting device 14 is configured to search for an available network among networks such as, for example, 2G, 3G, 4G, Wi-Fi, GSM, CDMA, GPRS, EDGE, EVDO, and/or HSPD. The application hosting device 14 also includes network module 40 configured to enable the application hosting device 14 to communicate with the data processing server 16 via any of the available networks.

The modules or components above may be implemented using any combination of software, firmware, and/or hardware. It is contemplated that other embodiments of the application hosting device 14 are not limited to the modules and components described above, and may include other modules or components.

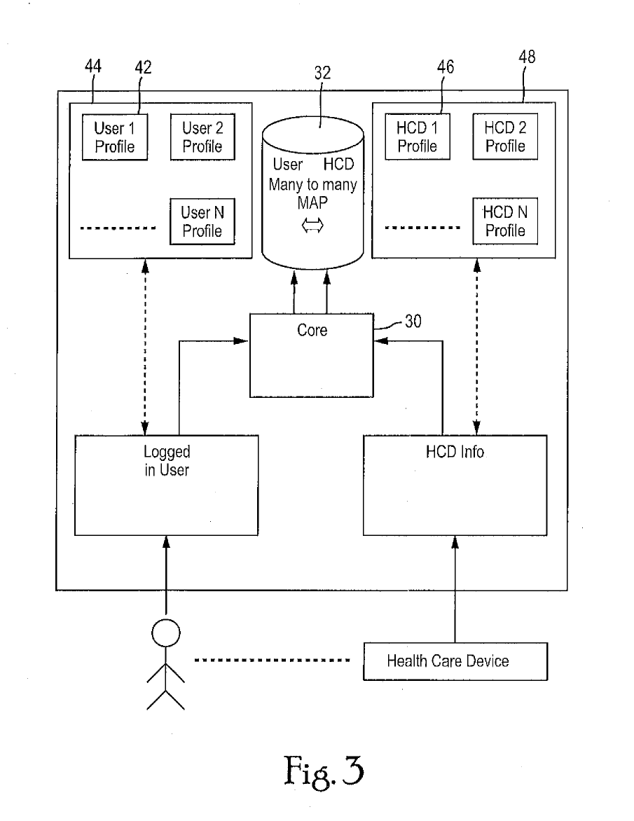

FIG. 3 shows an embodiment of the application hosting device 14 that may be used with multiple users and/or multiple medical devices 12. The application hosting device 14 may be configured to host programs or applications that may be implemented via a combination of software, firmware, and/or hardware. As such, the application hosting device 14 may provide user monitoring, medical device monitoring, user diagnostics, medical device updates, medical device programming, user data analysis, medical device data analysis, and/or other functions associated with the user and/or a medical device 12.

As mentioned above, the application hosting device 14 may be in communication with a medical device 12. In some embodiments, the application hosting device 14 is configured to be able to combine a voice network with several data networks to create a simultaneous multi-mode communications network. In other words, a user can use the application hosting device 14 for a telephone call or SMS text via a network, such as a telecommunications network, while data can be transmitted or received over any personal area network, such as the ones mentioned above, including Bluetooth, Zigbee, or NFC. In one embodiment, the application hosting device 14 can receive or send a telephone call or SMS text when the application hosting device 14 is transmitting and receiving information to and from the data processing server 16. In one embodiment, the application hosting device 14 is configured to combine up to four different communication networks. The application hosting device 14 may be configured to search for and select multiple networks based on availability. As such, the application hosting device 14 may be capable of using multiple connections to receive and transfer data from the medical device 12 via PAN to the data processing server 16 via LAN or WAN while simultaneously allowing, for example, voice communication. In other words, the application hosting device 14 can enable voice transmission via a telecommunication network, transfer of data via a PAN, and transfer of data via a WAN to occur simultaneously. In one embodiment, the system 10 includes PAN to WAN translation schemes for data originating from a medical device 12 so that the data can be received using a PAN and sent via a WAN. The system 10 may use protocol conversion to achieve flexibility and interoperability. In such embodiments, because the various networks used in the system 10 may be incompatible, the capability of the application hosting device 14 to receive data from one network and transmit the data over another network enhances the flexibility of the system 10.

The application hosting device 14 may enable asynchronous communication using an operating system's event mechanism thus leveraging multithreading capabilities. The application hosting device 14 may use the multihoming capabilities of the application hosting device 14 to maintain the availability of the communication channels such that multiple communication channels may be used simultaneously.

In one embodiment, the data packet received from a medical device 12 may initiate communication (e.g., via PAN or other communication network) between the medical device 12 and the application hosting device 14. If the user initiates a voice call, the application hosting device 14 may open a voice channel to accommodate the voice transmission. Simultaneously, the application hosting device 14 may receive data from the medical device 12 and may store the data. The application hosting device 14 may then upload the data to the server 16. The server 16 may send messages and/or instructions to the application hosting device 14 (e.g., via WAN or other communication network). The application hosting device 14 may then relay these messages and/or instructions to the medical device 12 via PAN. The voice transmission may occur at the same time as the data transfer. When data transfer is completed, the application hosting device 14 may operate in voice transmission only mode. Similarly, if the voice transmission is completed, the application hosting device 14 may operate in data transfer only mode.

As shown in FIG. 3, each user may have a user profile 42 stored in a user profile module 44 in the application hosting device 14. The user profile 42 may include data pertaining to a user such as user ID, password, name, address, phone numbers, nationality, social security number, date of birth, doctor's name, and/or other information. The user profile module 44 may be part of the memory of the application hosting device 14. In one embodiment, the user profile 42 may be stored in a database. The application hosting device 14 may also include a medical device profile 46 for each medical device 12 to which the application hosting device 14 connects. The medical device profile 46 may include data pertaining to a medical device 12 such as the device make; the device model number, the device serial number, and/or other information. The medical device profile 46 may be stored in a medical device profile module 48 in the application hosting device 14. The medical device profile module 48 may be part of the memory of the application hosting device 14. In one embodiment, the medical device profile module 48 may be a database in which the medical device profile 46 is stored as an entry.

Users may be able to register to use the system by inputting their information to be stored as a user profile 42. In one embodiment, the user may register and input user information into the application hosting device 14 using the user interface. In one embodiment, the information may be transmitted to the data processing server 16 from the application hosting device to be stored in the data processing server 16. Alternatively or additionally, the user may input and submit the user information to the data processing server 16, which then sends the user information for the user profile 42 to the application hosting device 14.

The medical device information for the one or more medical device profiles 46 may be manually inputted by the user, may be automatically retrieved from the medical device(s) 12, and/or retrieved from the data processing server 16. In one embodiment, the application hosting device 14 may automatically retrieve the information from the registry of the medical devices 12.

As mentioned above, the system 10 is capable of handling multiple users and multiple medical devices 12 linked with one another in various configurations, such as “one to many” or “many to many” (see FIGS. 14A-14C). Accordingly, any user linked to an application hosting device 14 can use any medical device 12 linked to an application hosting device 14 such that data can be transmitted to the server 16 for processing. To implement this process, the application hosting device 14 is able to support and store multiple user profiles 42 (as mentioned above with respect to FIG. 3). The user profiles 42 may contain information such as user ID and password. As mentioned above, the multiple medical devices 12 may be registered in the application hosting device 14 and the information corresponding to the medical devices 12 are stored in medical device profiles 46. The medical device profiles 46 may contain information such as device ID, device make, device model, and/or other information. As mentioned above, the application hosting device 14 maintains a mapping of a user profile with a medical device profile in database module 43. Therefore, a user may use multiple medical devices 12, multiple users may use a single medical device 12, or multiple users may use multiple medical devices 12. When a user uses the application hosting device 14, the user must log in using data from the user profile 42 (e.g., the user ID and password). The health care provider or other user may optionally select the user's profile to log in the user. After the user has logged in and the application hosting device 14 has identified the user, all of the data received by the application hosting device 14 from the medical device 12 is assumed to be associated with the user. When the data, such as vital sign or other measurement readings, is received from the medical device 12, the information from the device 12 is retrieved from the medical device profile 46. The information from the user profile 42 and from the medical device profile 46 are then aggregated, combined, or appended together with the data from the medical device 12 (such as the vital sign or other measurement readings) to be sent to the server 16 for further processing Thus, the application hosting device 14 may be configured to format or modify the data before sending the data to the server 16 for further processing

In one embodiment, the application hosting device 14 may serve as a hub such that information from one or more medical devices 12 can be transmitted automatically to the data processing server 16. The user may input their information when logging into the system 10. The medical device 12 information may be retrieved from the medical device 12. The application hosting device 14 may process, the information using the core logic module 30 so that the user can be associated with the medical device 12. The user to medical device 12 mapping may be stored in a database 43, which may be a relational database. It is contemplated that the mapping information may be stored in other forms of memory and in various formats.

After the user has logged into the system 10 using the application hosting device 14, the data sent from a medical device 12 may be received by the application hosting device 14, which stores the data and then automatically forwards the data, including any modification thereof, to the data processing server 16. It is contemplated that this process may be performed automatically without user intervention. By connecting to the medical device 12 and obtaining or retrieving the information from the medical device 12 automatically (the “auto handshake and auto connect” feature) and forwarding the information automatically, the system 10 preserves data integrity and ensures non-repudiation of data and non-repudiation of origin. In some embodiments, the application hosting device 14 may forward data to the server 16 as soon as the medical device 12 reads or obtains the data. Alternatively or additionally, the application hosting device 14 or the medical device 12 may optionally store the data for an amount of time before forwarding the data to the server 16 or to the application hosting device 14, respectively. For example, if the server 16 and the application hosting device 14 are unable to connect, the application hosting device 14 may store the data and forward the data when the connection is successful. As another example, if the application hosting device 14 and the medical device 12 are unable to connect, the medical device 12 may store the data and the application hosting device 14 may then retrieve the data from the medical device 12 once connection is successful. In some embodiments, the application hosting device 14 may be configured to store and forward at certain times, which may be determined by the user.

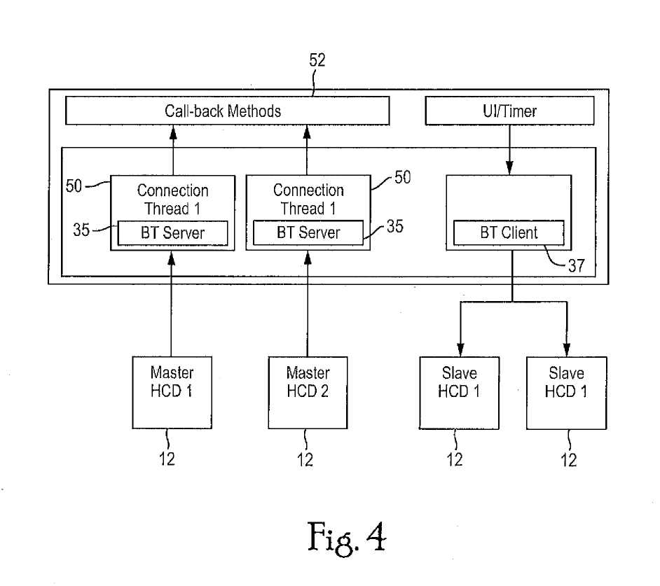

FIG. 4 shows an embodiment of the application hosting device 14 that may be capable of communicating simultaneously with multiple medical devices 12 by simultaneously interfacing to master and slave medical devices 12 to capture data and synchronously or asynchronously transfer data. The medical devices 12 may be classified as either a master device or a slave device according to their connection mechanisms using a PAN network, which may include Bluetooth, ZigBee, NFC, or others. A master medical device 12 initiates the connection between the application hosting device 14 and the medical device 12 when data (e.g., measurement data) is ready to be sent to the application hosting device 14. A slave medical device 12 stores the data (e.g., measurement data) in its memory and waits for the application hosting device 14 to connect to the slave medical device 12 and to request this data.

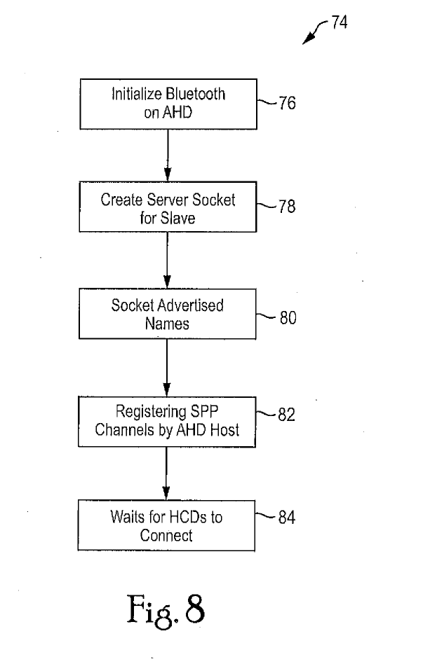

In the embodiment shown in FIG. 4, the application hosting device 14 is designed using multithreading to be able to simultaneously connect to multiple master and slave devices 12. In such an embodiment, a connection thread 50 is separately created for each master medical device 12 to communicate with the medical device 12. The thread initiates a server connection socket 35 and advertises a service for the medical device 12 in the service discovery protocol registry. The thread then waits for the device to initiate a connection via this socket 35. When this connection is initiated by the medical device 12, the thread accepts the connection and then waits for the medical device 12 to send data through the SPP channel. This data is received and the packets are sent to appropriate call back methods 52 registered in the device proprietary packet parsers module 32. This complete process is replicated in all of the threads 50 for all of the master medical devices 12. FIG. 8 illustrates this method and will be described in more detail later.

For a slave device, the application hosting device 14 opens a single socket 37 in client mode. Connection initialization is triggered on this socket 37 either by user interaction via the user interface or by a self repeating timer. In other words, the connection may be initialized based on the user's command or at intermittent times that may be programmed into the application hosting device 14. When the connection request is triggered, the application hosting device 14 sends a connection request to the medical device 12. In the case of, e.g., a Bluetooth arrangement, the application hosting device 14 would typically first search for the medical device 12 in its proximity and if the medical device 12 is in proximity, send the request If the connection is successful, the application hosting device 14 sends a request to the medical device 12 for, or else simply receives, the data (e.g., measurement data) or other information from the medical device 12. After the application hosting device 14 receives the data from the medical device 12, this information is then sent to the device proprietary packet parsers module 32 for further processing.

The system 10 may be capable of maintaining the identity and details of all medical devices 12 and users of the system 10. In one embodiment, the system 10 is able to track the path of the data from a medical device 12 to the data processing server 16 such that the system 10 is able to associate the data with a user, a medical device 12, and/or an application hosting device 14. In other words, the data can be traced back to the user, the medical device 12, and/or application hosting device 14.

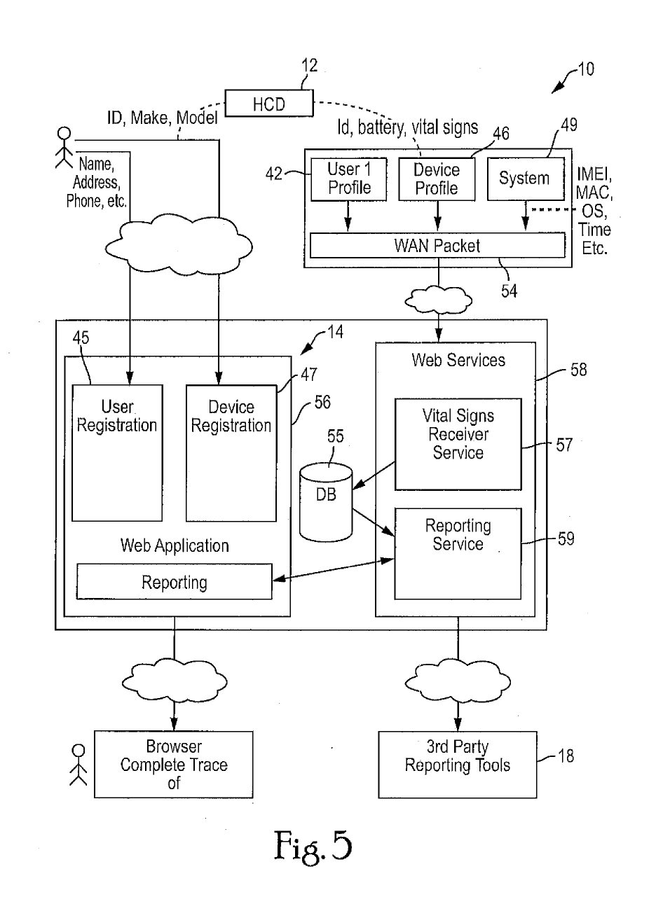

FIG. 5 shows an embodiment of the system 10 that enables traceability of data. As mentioned above, the application hosting device 14 stores information about the users and information about the medical devices 12 after the users and medical devices 12 have been registered. As shown in FIG. 5, the web application 56 includes user registration information 45 (e.g., information included in a user profile 42) and device registration information 47 (e.g., the information included in a medical device profile 46). Accordingly, the web application 56 is able to associate each user with a specific and identifiable medical device 12. The web application 56 may be hosted on the application hosting device 14. As such, the application hosting device 14 may gather information from the user profile 42, the medical device profile 46, and system profile 49 into a data packet 54, such as a WAN packet. The system profile 49 may contain info nation about the application hosting device 14, including, for example, the name of the device 14, the make and model number of the device 14, the MAC (Media Access Control) address, the operating system, the software version of the device 14, and a unique identifier associated with the application hosting device 14 (such as the IMEI number for a mobile device or the computer ID for a personal computer). The information listed is not intended to be limiting, and other information may be included. Before the user may upload data, the system 10 may require the user to enter user identification data, such as a password and user ID, so that the system 10 can identify the user. The packet 54 may therefore essentially embed authentication information, such as a user ID, password, and system information with the user's medical data (e.g., measurement readings) and/or other information (e.g., make, model, serial #, battery level, status, configuration, calibration, password, security key, encryption mode, etc.) about medical device 12. After this information has been gathered into a packet 54, the packet is transmitted to the web services API 58. Using the information from the packet 54, the system 10 is able to determine the complete path of the data from the user to the medical device 12 to the application hosting device 14 and to the server 16. Thus, using the data appended with the user information, the medical device information, and the application hosting device information, the system 10 is able to trace every byte of the data back to its origin. The web services API 58 may be operatively connected to a database 55 to store and retrieve the information from the packets 54 and may then transmit the data to third party applications, which will be described in more detail later. As shown in FIG. 5, a vital signs receiver service 57 is operatively connected to the database 55 to store vital signs information or other user measurement information. A reporting service 59 retrieves information from the database 55 for delivery to third party applications 18.

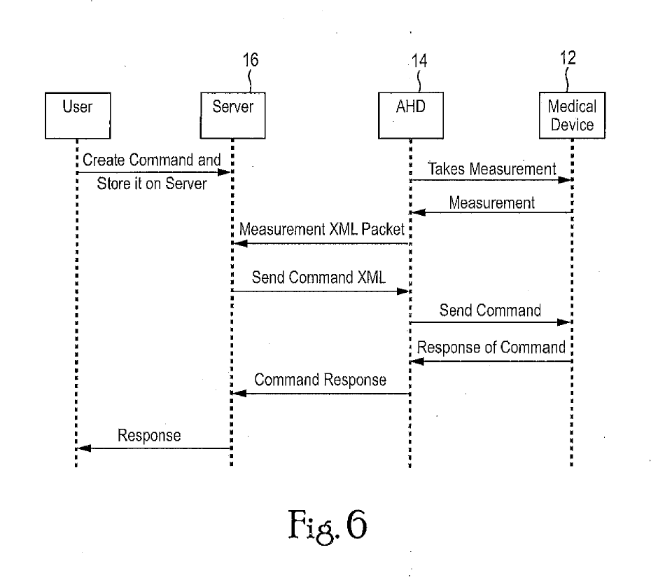

FIG. 6 is an exemplary signaling diagram illustrating a method of providing bi-directional communication. This bi-directional communication between the a medical device 12 and the application hosting device 14, between the application hosting device 14 and the data processing server 16, and between the data processing server 16 and a user enables a medical device 12 to be configured from the data processing server 16 and/or the application hosting device 14. In one embodiment, the system 10 may implement nodes. For example, one node reflects a set of configuration parameters for a medical device 12. Values can be read and parameters can be set using this node. Another node reflects the run-time environment for software applications on a device. Installing, upgrading, or uninstalling software elements may be performed using this node.

The medical device 12 may respond to specific commands or instructions. These commands may enable the user to, for example, set device settings, such as the time, date, or other measurements settings, remotely. The server 16 may create an XML data packet based on the command and may store the data packet in a database before transmitting the data packet to the application hosting device 14. As shown in FIG. 6, after the medical device 12 has obtained, for example, a user measurement or reading and has sent the measurement or reading to the application hosting device 14, the application hosting device 14 uploads the measurement or reading, along with user data, medical device data, and application hosting device data, to the server 16. Simultaneously or at around the same time, the server 16 checks for pending XML data packets to be sent to the application hosting device 14 to be relayed to the medical device 12 associated with the, application hosting device 14. After receiving the XML data packets from the server 16, the application hosting device 14 converts the XML data packets from the server 16 to the specific format for the medical device 12 and then sends the data to the medical device 12. The medical device 12 then performs the commands set forth in the XML data packets from the server 16. After performing the commands or instructions, the medical device 12 sends a response to the application hosting device 14 that the command has been performed. The application hosting device 14 then converts that response to XML format and sends the response to the server 16. Accordingly, the system 10 allows a user or third party to interact with (e.g., control, update, etc.) a medical device 12 remotely using the server 16 and the application hosting device 14. This enables an administrator or other health care provider to manage, update, and control one or more medical devices 12 remotely and efficiently. It is contemplated that the transmission of data between the server 16 and the application hosting device 14 may be in a variety of formats. It is also contemplated that the XML data packets may be sent at the user's request or at selected time intervals programmed in the system 10. In some embodiments, the XML data packets may be sent automatically as soon as they are created. In some embodiments, the commands may be stored on the application hosting device 14. That is, the user may input the commands into the application hosting device 14 and the application hosting device 14 may then format the instructions or commands for transmission to the medical device 12.

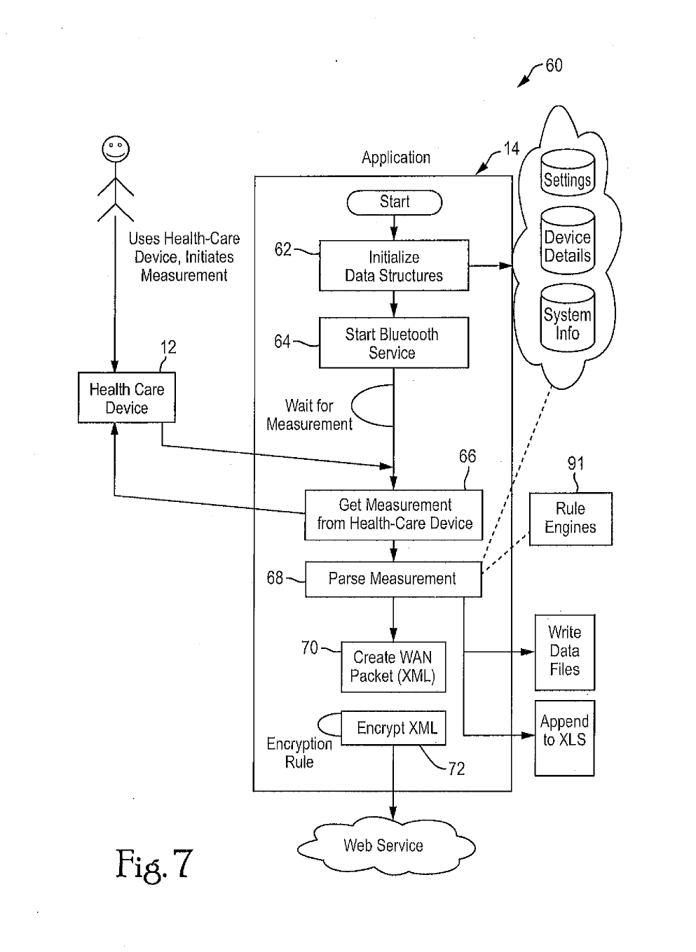

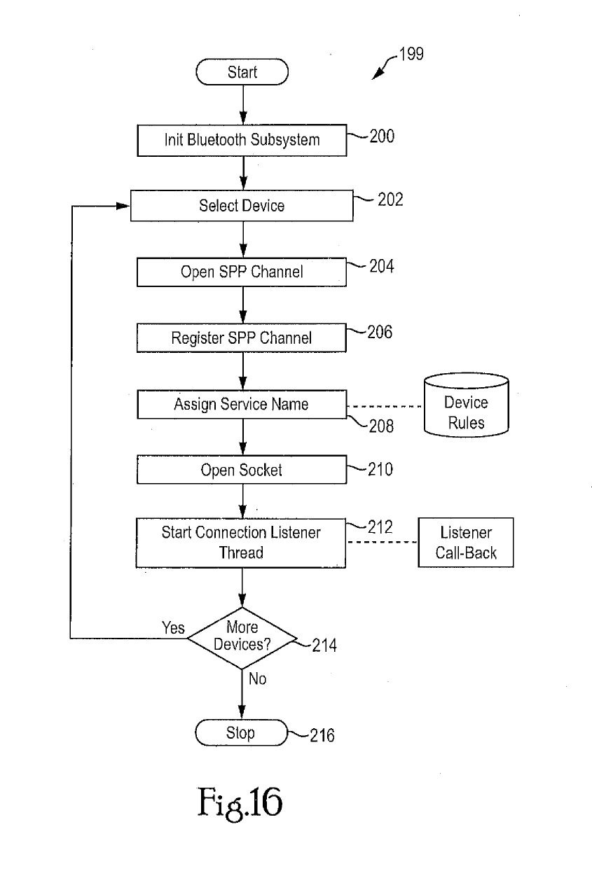

FIG. 7 is a flowchart illustrating a method of capturing data from a medical device 12. The process 60 starts at step 62 where the application on the application hosting device 14 is initialized. In step 62, the data structures for settings and system information may be initialized. The settings may include information such as user authentication info, device IDs, storage file names and paths, and other setting information. These settings may be stored as encrypted files. The system information may include information that is used by the system 10 to trace data through its complete traversal path (the traceability feature). The application hosting device 14 may send this information to the server 16 with the data obtained from the medical device 12. This system information may include the operating system name, version, hardware ID, and other system information of the application hosting device 14. The process 60 then proceeds to step 64 where a Bluetooth or any other network service is initialized. In this embodiment, the Bluetooth subsystem 34 is initialized so that the medical device 12 can communicate with the application hosting device 14. FIG. 8 illustrates in more detail a process 74 that may be implemented to initialize the Bluetooth subsystem 34. The Bluetooth RFCOMM (radio frequency communication) connection may be initialized on the application hosting device 14 as a master or client at step 76. In step 78 of process 74, a socket is created so that a slave medical device 12 can connect to the application hosting device 14 via a socket. A server socket is created so that a master medical device 12 can connect to the application hosting device 14 via the socket. The process 74 then proceeds to step 80 where the socket(s) has an advertised name for the medical device 12 to establish a connection. In step 82, the application hosting device 14 hosts the connection(s) by registering the SPP channel in the Service Discovery Protocol (SDP) records of the Bluetooth subsystem 34. The process 74 then proceeds to step 84 where the process 74 waits for the medical device 12 to connect. In an embodiment, the address and name of the medical device 12 is obtained and stored to avoid a time consuming discovery process. In an embodiment, the system 10 utilizes configuration files to establish the communication settings between the application hosting device 14 and the medical device 12. FIG. 16 is a flowchart that illustrates how master and slave devices may be connected to the application hosting device 14. The process 199 starts at step 200 where, for example, a Bluetooth subsystem 34 is initialized as master or client. In step 202, a medical device 12 is selected. The process 199 proceeds to step 204 where a SPP channel is opened. The SPP channel is then registered in the SDP records of the Bluetooth subsystem 34 in step 206. The process 199 then proceeds to step 208 where a service name is assigned. In some embodiments, a RFCOMM channel number can be specified. The process 199 then proceeds to step 210 where the socket is opened so that the application hosting device 14 can communicate with the medical device 12. The process 199 then proceeds to step 212 where a connection listener thread is started to listen for a client connection (e.g., a medical device connection). The listener thread may be used to wait for a client connection request and fork a thread to handle the connection. The listener thread may be used to manage an orderly shutdown of a client when a shutdown method is called. During this step, the system 10 may employ a listener callback function executed when notification of an event is received by the listener. The process 199 then proceeds to step 214 where the system 10 determines if there is more than one medical device 12. If there is a further device 12 to be connected, the process 199 proceeds back to step 202 to select another device 12. If there is no further device 12, the process 199 ends at step 216.

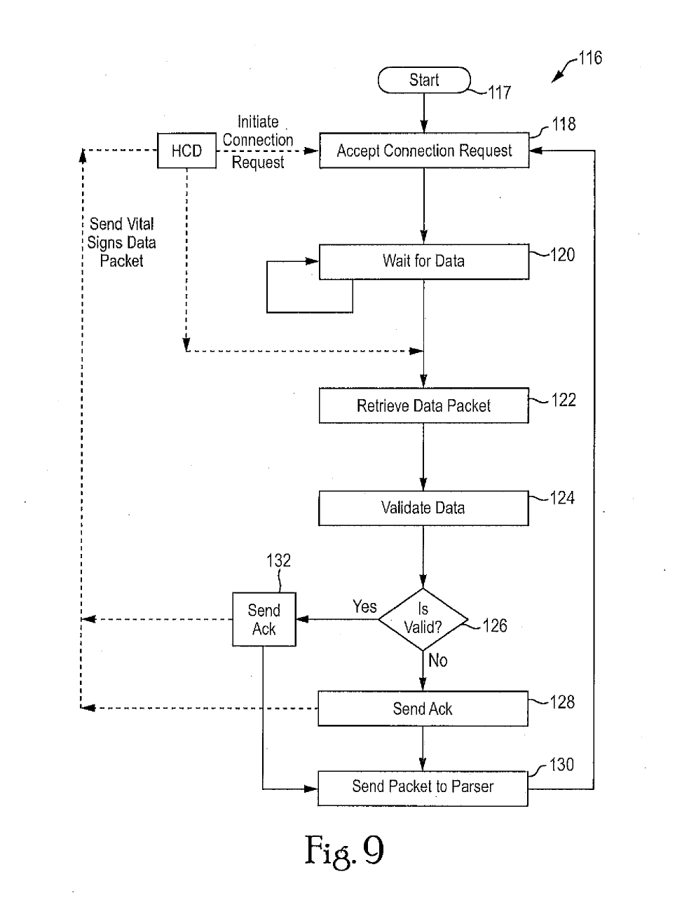

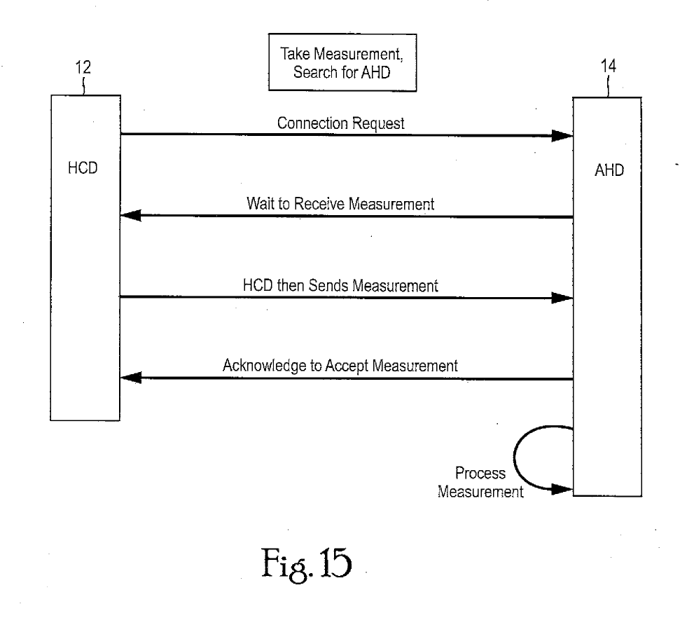

Referring back to FIG. 7, the process 60 proceeds to step 66 where the application hosting device 14 waits for data (e.g., a measurement) from the medical device 12 and eventually receives the data. FIG. 9 shows a flowchart that illustrates in more detail a method of communicating between the medical device 12 and the application hosting device 14 such that the application data hosting device 14 can receive data from the medical device 12. The process 116 starts at step 117 where after taking, for example, a measurement, the medical device 12 will search for the application hosting device 14 in its Bluetooth proximity. When the medical device 12 finds an application hosting device 14, the medical device 12 sends a connection request to the application hosting device 14 on the appropriate channel. In step 118, the application hosting device 14 accepts the connection request from the medical device 12. The process 116 then proceeds to step 120 where the application hosting device 14 has accepted this connection and is waiting for the data from the medical device 12. In step 122, the medical device 12 sends the data in a proprietary or standard format over the Bluetooth SPP channel. In step 124, the application hosting device 14 validates the data as being in a correct format. In step 126, the application hosting device 14 determines whether the data is valid, such as whether the data is in a correct format. If the data is valid, the process 116 proceeds to step 132 where the application hosting device 14 sends an acknowledgement to the medical device 12. The process 116 then proceeds to step 130 where the data is parsed. If the data is not valid, the application hosting device 14 sends a response to the medical device 12 to inform the medical device 12 that the data is not valid. At that point, the medical device 12 may again send data to the application hosting device 14. FIG. 15 is a signaling diagram showing communication between the medical device 12 and application hosting device 14 described above with respect to FIG. 9. In this embodiment, the medical device .12 requests a connection with the application hosting device 14, the application hosting device 14 waits for the data (e.g., measurement data) from the medical device 12, the medical device 12 then sends the data to the application hosting device 14, and the application hosting device 14 then sends an acknowledgement or response to the medical device 12 after the application hosting device 14 has successfully received the data.

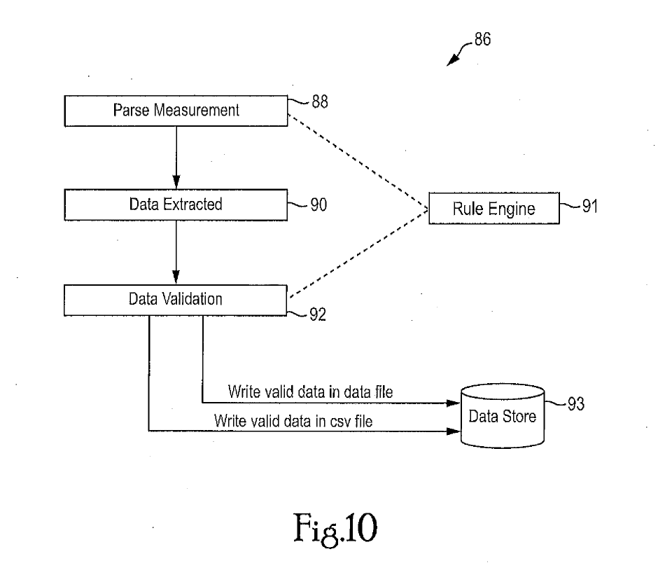

Referring back to FIG. 7, the process 60 then proceeds to step 68 where the application hosting device 14 parses the data (e.g., measurement). In this step 68, the data received from the medical device 12 is parsed using a rules engine for that particular medical device 12. FIG. 10 shows in more detail a process 86 for parsing the data, such as measurements. In step 88 of process 86, the application hosting device 14 parses the data received from the medical device 12. In step 90, information such as measurement values, units, time of measurement, device information, battery strength, and/or other information is retrieved or extracted from the data. The process 86 then proceeds to process 92 where the extracted information is checked for validity using a rule engine 91. The extracted information is then stored in a data store 93, which may be part of the data storage 22 of the application hosting device 14. In one embodiment, raw data is stored as is (without formatting) in a file. In an embodiment, measurement values are stored in a csv (comma separated value) file for easy viewing in a spreadsheet application (e.g., Microsoft Excel). Data may be stored in a plain text file for easy viewing on the application hosting device 14.

Referring back to FIG. 7, the process 60 then proceeds to step 70 where the packet 54 (e.g., a WAN packet) is created for uploading to the server 16. Using the medical device data, the system information of the application hosting device 14, and information of the medical device 12, a data packet 54 is created. In one embodiment, the data packet 54 is created in XML form, although it is contemplated that another format may be used. Authentication information of the user may be added to the packet 54. The process 60 then proceeds to step 72 where the packet 54 is encrypted using any suitable encryption technique or rule. The encrypted packet 54 is sent via, for example, the Internet using the application hosting device 14's available connection. As mentioned above, the application hosting device 14 is configured to search for any available network by which it can connect to the server 16. The application is written to seamlessly send the packet 54 to the server 16 without disrupting any of the ongoing network activity on the application hosting device 14. When the server 16 has received the packet 54, the server 16 acknowledges the packet 54 and the process 60 is completed. If, however, the packet 54 cannot be uploaded due to any communication failure or any other reasons, the data packet 54 is queued, cached, or tagged to be sent whenever the connection is available.

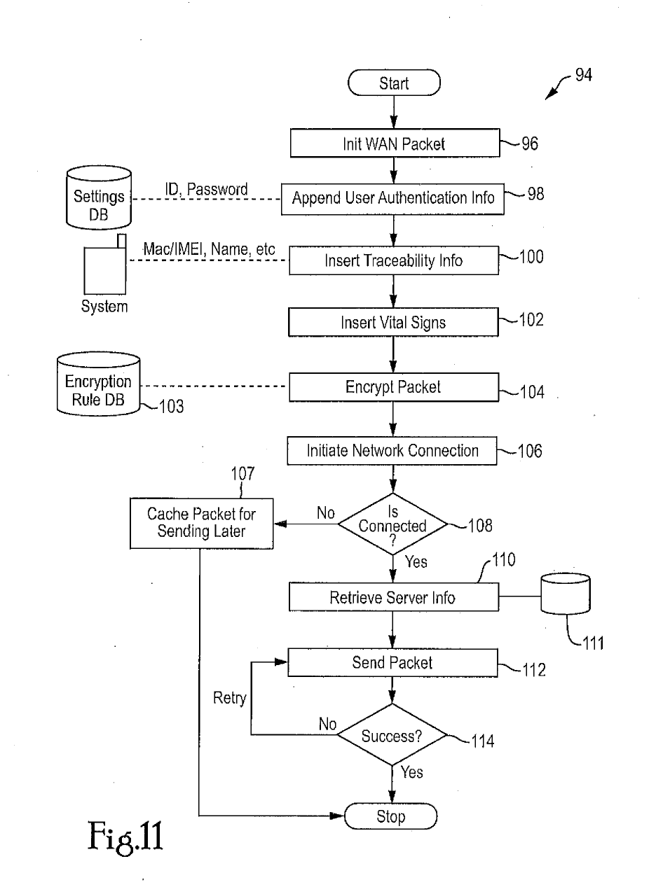

FIG. 11 shows a detailed method of creating a data packet 54 for uploading to the server 16. The process 94 starts at step 96 where the data packet 54 is initialized. The process 94 proceeds to step 98 where user authentication information, such as a User ID and password, is included in the data packet 54. The process 94 then proceeds to step 100 where traceability information is inserted into the data packet 54. As mentioned above, traceability information may include the application hosting device 14's system information, such as Mac/IMEI, the name of the application hosting device 14, and/or other information that may help identify the application hosting device 14 The process 94 then proceeds to step 102 where the medical device data (e.g., vital signs or other measurement data) is inserted into the data packet 54. The process 94 proceeds to step 104 where the data packet 54 is encrypted using an encryption rule stored in a database 103. The process 94 proceeds to step 106 where a network connection is initialized. The network connection may be initialized using any of the methods mentioned above. After the network connection has been initialized, the process 94 proceeds to step 108 where the process 94 determines if the application hosting device 14 is connected to the server 16. If the application hosting device 14 is not connected to the server 16, the process 94 proceeds to step 107 where the data packet 54 is cached for transmission later. If a connection has been established, the server 16 information is retrieved from a database 111 or from the server 16 so that the application hosting device 14 can transmit the data packet 54 to the server 16 in step 112. In step 114, the process 94 determines whether the transmission was successful. If so, the process 94 ends. If the packet 54 was not successfully sent, step 112 is repeated until the data packet 54 is successfully sent.

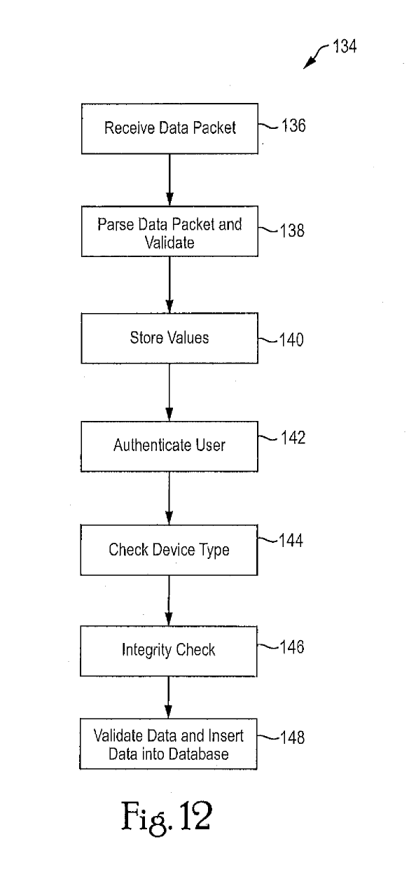

FIG. 12 is a flowchart illustrating a method of processing a data packet 54 received from the application hosting device 14. The process 134 starts at step 136 where the data packet is received from the application hosting device 14. The data packet 54 may be sent from the application hosting device 14 via the HTTP protocol. If the data packet 54 is encrypted, the data packet 54 is decrypted using a decryption rule. If the data packet 54 is in a XML format, the XML schema is compared. The process 134 then proceeds to step 138 where the data is parsed. The data packet 54 may be a standard format, for example, PCD-01 (HL v2.6). If the data packet 54 is in XML format, then the XML is parsed using, for example, a XML DOM parser. In one embodiment, the XML string is parsed and looped through xml nodes so that the values may be fetched and saved in an array string in step 140, although it is contemplated that the values may be saved in other forms. The following information may be retrieved and stored: user authentication information (e.g., user ID, password, or other information), medical device information (e.g., device ID, battery level, or other information), and/or application hosting device 14 system information (e.g., IMEI/MAC address, system name, or other information). The medical device data (e.g., measurement data) is also retrieved and stored. If the parsing is successful, the process proceeds to step 142. Otherwise, an error code is returned to the application hosting device 14. In step 142, the server 16 authenticates the user. The authentication information is retrieved from the data packet 54. The authentication is performed using the user ID and the password obtained from the user table in a database 55 (see FIG. 5) associated with the server 16. The user ID and password from the database is compared with the XML credentials. In one embodiment, the password may be encrypted using a MD5 algorithm resulting in a 32-digit hexadecimal number. This hexadecimal number is compared with the hexadecimal number stored in the database 55. If the authentication fails, the resulting error message may be saved in an error table in the database 55. If the authentication is successful, the process 134 then proceeds to step 144 where the device type is checked to determine what type of medical device 12 submitted the data. For example, the device type might be a blood pressure monitor, a weight scale, a glucose monitor, or other device. The process 134 then proceeds to step 146 where the server 16 determines if the device ID in the data packet 54 is associated with the user information provided from the user table in the database 55. If the user is already associated with the medical device 12, then the data is validated as being in the correct data format for insertion into the database and the validated data is inserted into the database in step 148. If not, an error code is returned to the application hosting device 14. In step 146, the server 16 determines if the medical device data is in the correct format and if so, inserted into the database in step 148. If not, an error code is returned to the application hosting device 14. The medical device data is stored in the appropriate table for that device type. For example, in one embodiment, measurement data obtained from a blood pressure device is stored in a blood pressure device records table in the database 55. The routing or traceability information (including the device ID, the application hosting device 14's system information, and other information) may also be stored in the database 55. In one embodiment, the medical device data retrieved from a data packet from the application hosting device 14 may be stored according to user. For example, after the server 16 has identified which user is associated with the medical device data, the server 16 may place the data for that user into a table in the database 55 specifically created for that user. It is contemplated that the data retrieved from the data packets 54 may be stored in a variety of ways and are not limited to the methods described above.

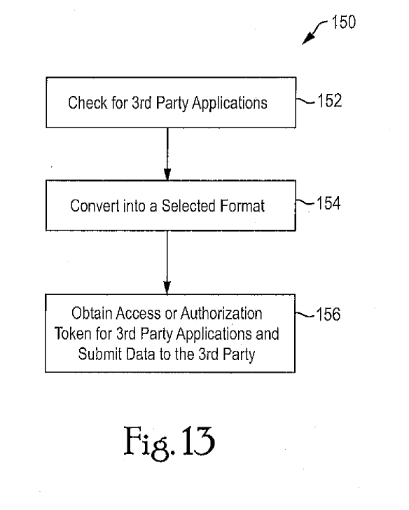

FIG. 13 illustrates a method for submitting data to one or more third party applications or systems 18. In step 152, the server 16 checks the user table in the database 55 to determine if the user allows the posting of data to a third party application or system. If so, the user's corresponding authorization or access token is retrieved from the database 55. For example, the user might allow data to be sent to Google Health, Microsoft Health Vault, Dossia, Twitter, Facebook, or other third party application. In some embodiments, the user might also allow data to be sent, for example via SMS, to a third party device (such as mobile device). If the user allows data to be sent, the data is converted into a selected format in step 154. For example, if Google Health, Microsoft Health Vault and/or Dossia is chosen as an allowed third party to receive the data, the data may be converted into ASTM Continuity of Care Record Standard (CCR). The server 16 may obtain the user's authorization or access token for the third party application and the CCR document may then be posted to the Google Health service via the Google Health API method or the CCR document may be sent to the Microsoft Health Vault or Dossia service. In some embodiments, the data may be sent to Twitter, Facebook, or via SMS. If the user has selected to update Twitter with the data, the server 16 may create a message. The access or authorization token may be retrieved and the message may be sent to Twitter using the Twitter API method. If the user has selected to send the data to Facebook, a message is created for posting on a Facebook wall. Variables, such as session_key and userID for Facebook, may be retrieved from the database 55, and using the Facebook API method, the Facebook wall may be updated or the user's status on Facebook may be updated with the information. If the user has selected to send the data via SMS, the server 16 determines if the user has inserted a mobile phone number. If a mobile number is in the database 55, a message is created to send via SMS and the mobile number is retrieved from the database 55. The SMS may then be sent through the http protocol. If any errors are returned by the third parties, the error code is forwarded to the application hosting device 14. If the data is successfully sent to the third parties, the success code is sent to the application hosting device 14.

It is contemplated that any of the features of the system 10 may be implemented using any combination of medical devices 12, application hosting devices 14, and servers 16. Additionally, the functions performed by each of the components of the system 10 may be performed using other components, and the features of each component mentioned above may be included in other components of the system 10. Although one server 16 is shown in the Figures, it is contemplated that the server 16 may comprise multiple servers 16. The multiple servers 16 may be operatively connected to one another.

Embodiments of the invention may be made in hardware, firmware, software, or various combinations thereof. An embodiment of the invention may be implemented as instructions stored on a machine-readable storage medium, which may be read and executed using one or more processing devices. In one embodiment, the machine-readable storage medium may include various mechanisms to store and/or transmit information in a form that can be read by a machine (e.g., a computing device For example, a machine-readable storage medium may include read only memory, random access memory, magnetic disk storage media, optical storage media, flash memory devices, or other storage media. While firmware, software, routines, or instructions may be described in the above disclosure in terms of specific exemplary aspects and embodiments performing certain actions, it will be apparent that such descriptions are merely for the sake of convenience and that such actions in fact result from one or more computing devices, processing devices, processors, controllers, or other devices or machines executing the firmware, software, routines, or instructions.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment. Furthermore, since numerous modifications and changes will readily occur to those of skill in the art, it is not desired to limit the invention to the exact construction and operation described herein. Accordingly, all suitable modifications and equivalents should be considered as falling within the spirit and scope of the invention.

What is claimed is:

1. A system for monitoring and controlling medical devices, comprising:
a data processing server configured to receive from a user instructions relating to a medical device; and
an application hosting device configured to receive the instructions from the data processing server, the application hosting device further configured to modify the instructions for transmission to the medical device,
wherein the medical device is configured to receive the modified instructions and perform the modified instructions.

2. The system of claim 1, wherein the application hosting device is configured to convert the instructions to a selected format useable by the medical device.

3. The system of claim 1, wherein the instructions comprise updates relating to the medical device settings.

4. The system of claim 1, wherein the instructions comprise an identifier identifying the medical device to receive the modified instructions from the application hosting device.

5. A system for delivering medical device data, comprising:
an application hosting device configured to receive data from a medical device using a first network, the application hosting device being further configured to process the data and send the data via a second network, wherein the first network is different from the second network.

6. The system of claim 5, wherein the first network is a personal area network.

7. The system of claim 5, wherein the second network is a wide area network.

8. The system of claim 5, wherein the second network is a local area network.

9. The system of claim 5, wherein the application hosting device is capable of multi-mode connection.

10. The system of claim 9, wherein the application hosting device is configured to simultaneously enable voice transmission when receiving data from the first network and when sending data via the second network.

11. The system of claim 5, wherein the application hosting device stores the data received from the medical device in a memory.

12. An application hosting device to capture medical device data, the application hosting device including a processor and a connection unit-configured to establish a link to exchange information, the application hosting device being configured to connect to a plurality of medical devices simultaneously, wherein the plurality of medical devices comprise at least one master device and at least one slave device.

13. The application hosting device of claim 12, wherein the link is a Bluetooth

14. The application hosting device of claim 12, wherein the application hosting device uses multithreading to connect to the plurality of medical devices simultaneously.

15. A method of monitoring and controlling a medical device, comprising:
receiving, from a data processing server, instructions relating to a medical device;
identifying the medical device associated with the instructions;
modifying the instructions for transmission to. the medical device; and
transmitting the modified instructions to the medical device.

16. The method of claim 15, wherein modifying the instructions comprises converting the instructions into a format useable by the medical device.

17. The method of claim 15; wherein the instructions comprise updates relating to the medical device settings.

18. A computer-readable storage medium having computer-executable code for execution by a processing system to control a medical device, the code configured to:
identify the medical device associated with received instructions;
modify the instructions for transmission to the medical device; and
transmit the modified instructions to the medical device.

19. The computer-readable medium of claim 18, wherein the code to modify the instructions comprises code to convert the instructions into a format useable by the medical device.

20. The computer-readable medium of claim 18, wherein the instructions comprise updates relating to the medical device settings.
